(19) United States
(12) Patent Application Publication
(10) Pub. No.: US 2023/0165658 A1
(43) Pub. Date: Jun. 1, 2023

(54) SYSTEMS AND METHODS FOR CONNECTING A MEDICAL IMAGING DEVICE TO A MEDICAL IMAGING CONTROLLER

(71) Applicant: Stryker Corporation, Kalamazoo, MI (US)

(72) Inventors: William H. L. CHANG, Milpitas, CA (US); Chien Mien PANG, San Jose, CA (US); Ajay RAMESH, Pleasanton, CA (US); Danny NGUYEN, San Jose, CA (US)

(73) Assignee: Stryker Corporation, Kalamazoo, MI (US)

(21) Appl. No.: 18/059,969

(22) Filed: Nov. 29, 2022

Related U.S. Application Data
(60) Provisional application No. 63/284,603, filed on Nov. 30, 2021.

(51) Int. Cl. A61B 90/00; H04N 23/66

(52) U.S. Cl. CPC A61B 90/361; H04N 23/66

(57) ABSTRACT

An exemplary system for acquiring surgical imaging data comprises a surgical imaging device; an imaging controller; and a communication cable for connecting the surgical imaging device to the imaging controller, comprising: a distal end for connecting with the surgical imaging device; a proximal end for connecting with the imaging controller; a single conductor extending between the distal end and the proximal end of the communication cable, the single conductor configured to: transmit surgical imaging data from the surgical imaging device to the imaging controller, and transmit a control signal and power from the imaging controller to the surgical imaging device.

### CROSS-REFERENCE TO RELATED APPLICATIONS

This application claims the benefit of U.S. Provisional Application No. 63/284,603, filed Nov. 30, 2021, the entire contents of which are hereby incorporated by reference herein.

### FIELD

The present invention generally relates to medical imaging, and more specifically to connecting medical imaging devices to medical imaging controllers.

### BACKGROUND

Medical systems, instruments or tools are utilized pre-surgery, during surgery, or post-operatively for various purposes. Some of these medical tools may be used in what are generally termed endoscopic procedures or open field procedures. For example, endoscopy in the medical field allows internal features of the body of a patient to be viewed without the use of traditional, fully invasive surgery. Endoscopic imaging systems incorporate endoscopes to enable a surgeon to view a surgical site, and endoscopic tools enable minimally invasive surgery at the site. Such tools may be shaver-type devices which mechanically cut bone and hard tissue, or radio frequency (RF) probes which are used to remove tissue via ablation or to coagulate tissue to minimize bleeding at the surgical site, for example.

In endoscopic surgery, the endoscope is placed in the body at the location at which it is necessary to perform a surgical procedure. Other surgical instruments, such as the endoscopic tools mentioned above, are also placed in the body at the surgical site. A surgeon views the surgical site through the endoscope in order to manipulate the tools to perform the desired surgical procedure. Some endoscopes are usable along with a camera head for the purpose of capturing and processing the images received by the endoscope. An endoscopic camera system typically includes a camera head connected to a camera control unit (CCU) by a cable. The CCU processes input image data received from the image sensor of the camera via the cable and then outputs the image data for display. The resolution and frame rates of endoscopic camera systems are ever increasing and each component of the system must be designed accordingly.

Another type of medical imager that can include a camera head connected to a CCU by a cable is an open-field imager. Open-field imagers can be used to image open surgical fields, such as for visualizing blood flow in vessels and related tissue perfusion during plastic, microsurgical, reconstructive, and gastrointestinal procedures.

Existing imaging systems (e.g., endoscopic imaging systems, open-field imaging systems, and other types of medical imaging systems) rely on a cable having multiple conductors (i.e., multiple conductive wires) to transmit high-speed signals (e.g., image data), low-speed signals (e.g., control signals), and power over the multiple conductive wires. Such a multi-conductor cable suffers from a number of deficiencies. For example, when image data is transmitted via multiple conductive wires in the same cable, the system needs to synchronize the multiple streams of data. Synchronization can become more complex to implement as the number of conductive wires increases in the cable to support a higher data transmission rate. As the number of conductive wires increases, the cable also becomes more difficult and expensive to manufacture. The cable also becomes more prone to wear and tear and less environmentally friendly. Further, the multiple conductive wires in the same cable can cause interferences (e.g., crosstalk) among the transmitted signals and compromise the integrity of the signals. Further still, a cable having a specific number of conductive wires for separately transmitting the specific number of data streams can result in a complex interface and can be difficult to integrate into a variety of imaging systems.

### SUMMARY

Described herein are devices, systems, and methods for connecting an imaging device to an imaging controller with a single-conductor cable. The imaging systems described herein provide a superior, highly scalable infrastructure for high-speed image data transmission. The imaging system comprises a single-conductor cable used for transmitting image data (e.g., at around or over 12 gigabits per second), non-image data such as control signals (e.g., at around or over 40 megabits per second), and power. The single-conductor cable comprises only one conductor for transmitting image data, non-image data such as control signals, and power, and optionally a ground conductor, such as a shielding. The single conductor can be an electrical conductor (e.g., metal or metal alloy). In some examples, the system comprises an imaging device comprising a transmitter for driving the cable and an imaging controller comprising a receiver to perform equalization.

The single-conductor cable provides a number of technical advantages. For example, the single-conductor cable does not generate interferences that would otherwise occur in a multi-conductor cable. Further, the single-conductor cable does not require synchronization among multiple conductors, thus resulting in a simplified and more robust hardware and software assembly. The single-conductor cable and the interfaces for the cable are easier to manufacture, to use, and to maintain comparing to a multi-conductor cable assembly. The single-conductor cable is smaller in diameter and more pliable, making it less susceptible to wear and tear and to environmental factors (e.g., moisture, sterilization). The single-conductor cable also requires less material and is longer lasting and more environmentally friendly. Numerous other technical improvements of the examples are described in detail herein.

According to an aspect is provided a system for acquiring surgical imaging data comprising a surgical imaging device, an imaging controller, and a communication cable for connecting the surgical imaging device to the imaging controller. The communication cable comprises a distal end for connecting with the surgical imaging device, and a proximal end for connecting with the imaging controller. The communication cable comprises a single conductor extending between the distal end and the proximal end of the communication cable. The single conductor is configured to transmit surgical imaging data from the surgical imaging device to the imaging controller, and transmit a control signal and power from the imaging controller to the surgical imaging device.

Optionally, the surgical imaging data comprises at least one of pixel data and voxel data.

Optionally, the surgical imaging data comprises data acquired by a plurality of sensors of the surgical imaging device.

Optionally, the control signal is a first control signal, and the single conductor is further configured to transmit a second control signal from the surgical imaging device to the imaging controller.

Optionally, the surgical imaging data, the control signal, and the power are transmitted according to CoaXPress protocol.

Optionally, the single conductor is configured to transmit the surgical image data at around or over 12 gigabits per second.

Optionally, the single conductor is configured to transmit the control signal at around or over 40 megabits per second.

Optionally, the single conductor is insulated, the communication cable further comprising a cable shielding wrapped circumferentially around the insulated single conductor.

Optionally, the cable shielding is configured to provide ground to the power.

Optionally, the single conductor is made of at least copper.

Optionally, the communication cable comprises a first connector at the proximal end.

Optionally, the communication cable comprises a second connector at the distal end.

Optionally, the distal end of the communication cable is connected to a transmitter of the surgical imaging device.

Optionally, the transmitter is configured to transmit the surgical imaging data according to the CoaXPress protocol.

Optionally, the transmitter includes a clock.

Optionally, the proximal end of the communication cable is connected to a receiver of the imaging controller.

Optionally, the receiver is housed in a connector board of the imaging controller.

Optionally, the receiver is configured to receive the surgical imaging data according to the CoaXPress protocol.

Optionally, the receiver is configured to perform equalization on the received surgical imaging data.

Optionally, the communication cable is a first cable and the surgical imaging data is a first set of surgical imaging data, and the system further comprises: a second cable for connecting the surgical imaging device to the imaging controller, wherein the second cable is configured to transmit a second set of surgical imaging data from the surgical imaging device to the imaging controller.

According to an aspect is provided a surgical imaging device configured to be connected to a communication cable. The surgical imaging device comprises a camera component for obtaining surgical imaging data, and a transmitter configured to be connected to the communication cable. The transmitter is configured to simultaneously transmit the surgical imaging data via a single conductor of the communication cable, and receive a control signal and power via the single conductor of the communication cable.

Optionally, the transmitter is configured to transmit the surgical imaging data according to the CoaXPress protocol.

Optionally, the transmitter includes a clock.

Optionally, the control signal is a first control signal, and the transmitter is configured to transmit a second control signal via the single conductor of the communication cable.

According to an aspect is provided an imaging controller configured to be connected to a communication cable. The imaging controller comprises a control component for generating a control signal, and a receiver configured to be connected to the communication cable. The receiver is configured to simultaneously receive surgical imaging data via a single conductor of the communication cable, and transmit the control signal and power via the single conductor of the communication cable.

Optionally, the receiver is housed in a connector board of the imaging controller.

Optionally, the receiver is configured to receive the surgical imaging data according to the CoaXPress protocol.

Optionally, the receiver is configured to perform equalization on the received surgical imaging data.

Optionally, the control signal is a first control signal, and the receiver is configured to receive a second control signal via the single conductor of the communication cable.

According to an aspect is provided a method for acquiring surgical imaging data from a surgical imaging device to an imaging controller, via a communication cable connecting the surgical imaging device to the imaging controller. The method includes having the communication cable connected at a distal end with the surgical imaging device, and at a proximal end with the imaging controller. The communication cable comprises a single conductor extending between the distal end and the proximal end of the communication cable. The method comprises transmitting surgical imaging data from the surgical imaging device to the imaging controller, and transmitting a control signal and power from the imaging controller to the surgical imaging device via the communication cable.

It will be appreciated that any one or more of the above aspects, features and options can be combined. It will be appreciated that any one of the options described in view of system apply equally to the imaging device, imaging controller or method, and vice versa.

### BRIEF DESCRIPTION OF THE FIGURES

The invention will now be described, by way of example only, with reference to the accompanying drawings, in which:

FIG. 1A shows an example of an endoscopic camera system;

FIG. 1B shows an example of an open-field camera system;

FIG. 2A and FIG. 2B illustrates an example of an imaging system that includes an imaging device connected to an imaging controller by a multi-conductor cable;

FIG. 3A and FIG. 3B illustrates an example of an imaging system that includes an imaging device connected to an imaging controller by a single-conductor cable;

FIG. 4 illustrates exemplary data rates supported by the imaging system;

FIG. 5 illustrates an exemplary imaging system operating according to the CoaXPress protocol;

FIG. 6 is an illustrative depiction of an exemplary fluorescence imaging system;

FIG. 7 is an illustrative depiction of an exemplary illumination module of a fluorescence imaging system; and

FIG. 8 is an exemplary camera module of a fluorescence imaging system.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are devices, systems, and methods for connecting an imaging device to an imaging controller with a single-conductor cable. In existing imaging systems, a cable having multiple conductors (i.e., multiple conductive wires) is used to transmit high-speed signals (e.g., image data), low-speed signals (e.g., control signals), and power over the multiple conductive wires. Such a multi-conductor cable suffers from a number of deficiencies. For example, when image data is transmitted via multiple conductive wires in the same cable, the system needs to synchronize the multiple streams of data. Synchronization can become more complex to implement as the number of conductive wires increases in the cable to support a higher data transmission rate. As the number of conductive wires increases, the cable also becomes more difficult and expensive to manufacture. The cable also becomes more prone to wear and tear and less environmentally friendly. Further, the multiple conductive wires in the same cable can cause interferences among the transmitted signals and compromise the integrity of the signals. Further still, a cable having a specific number of conductive wires for separately transmitting the specific number of data streams can result in a complex interface and can be difficult to integrate into a variety of imaging systems.

The imaging systems described herein provide a superior, highly scalable infrastructure for high-speed image data transmission. The imaging system comprises a single-conductor cable used for transmitting image data (e.g., at around or over 12 gigabits per second), non-image data such as control signals (e.g., at around or over 40 megabits per second), and power. In some examples, the system comprises an imaging device comprising a transmitter for driving the cable and/or an imaging controller comprising a receiver to e.g. perform equalization.

The single-conductor cable provides a number of technical advantages. For example, the single-conductor cable does not generate interferences that would otherwise occur in a multi-conductor cable. Further, the single-conductor cable does not require synchronization among multiple conductors, thus resulting in a simplified and more robust hardware and software assembly. The single-conductor cable and the interfaces for the cable are easier to manufacture, to use, and to maintain comparing to a multi-conductor cable assembly. The single-conductor cable is smaller in diameter and more pliable, making it less susceptible to wear and tear and to environmental factors (e.g., moisture, sterilization). The single-conductor cable also requires less material and is longer lasting and more environmentally friendly. Numerous other technical improvements of the examples are described in detail herein.

In the following description of the various examples, reference is made to the accompanying drawings, in which are shown, by way of illustration, specific examples that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

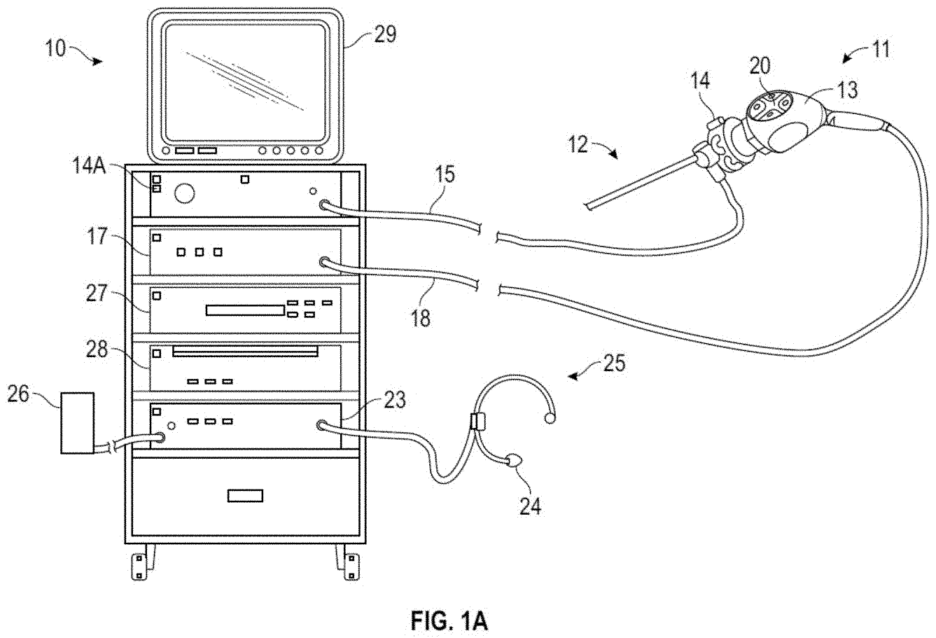

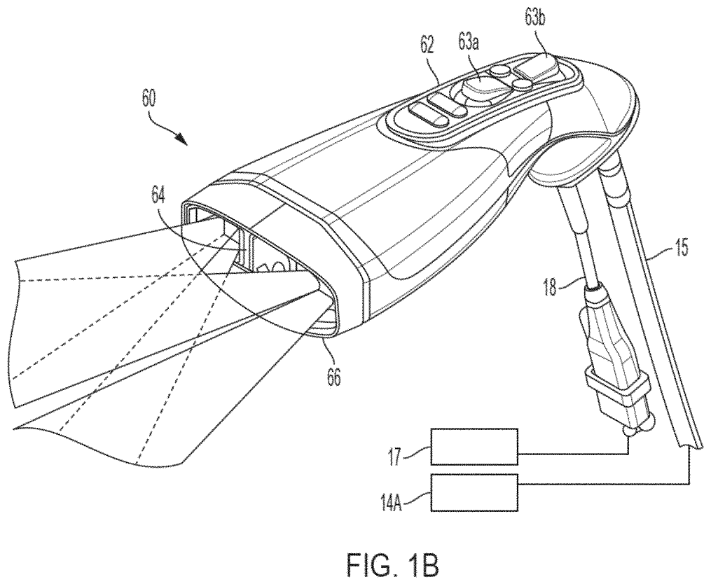

FIG. 1A shows an exemplary medical imaging system 10 that can utilize an, e.g. authenticable, data cable for connecting a medical imaging device to a medical imaging controller, according to the principles described herein. As used herein, medical imaging includes, but is not limited to, pre-operative, intra-operative, post-operative, and diagnostic imaging sessions and procedures. System 10 includes a scope assembly 11 which may be utilized in endoscopic procedures. The scope assembly 11 incorporates an endoscope or scope 12 which is coupled to an endoscopic camera head 13 by a coupler 14 located at the distal end of the camera head 13. Light is provided to the scope by a light source 14A via a light guide 15, such as a fiber optic cable. The camera head 13 is connected to a camera control unit (CCU) 17 by an electrical cable 18. Operation of the camera 13 is controlled, in part, by the CCU 17. The cable 18 conveys or transmits still and/or video image data from the camera head 13 to the CCU 17 and conveys various control signals bi-directionally between the camera head 13 and the CCU 17. In one example, the image data output by the camera head 13 is digital. The cable 18 may include a memory device for storing authentication data for authenticating the cable 18, as discussed further below.

A control or switch arrangement 20 may be provided on the camera head 13 and allows a user to manually control various functions of the system 10. These and other functions may also be controlled by voice commands using a voice-control unit 23, which is connected to the CCU 17. Optionally, voice commands are input into a microphone 24 mounted on a headset 25 worn by the surgeon and wiredly, or wirelessly, coupled to the voice-control unit 23. A hand-held control device 26, such as a tablet with a touch screen user interface or a PDA, may be connected to the voice control unit 23 as a further control interface. In the illustrated example, a recorder 27 and a printer 28 are also connected to the CCU 17. Additional devices, such as an image capture and archiving device, may be included in the system 10 and connected to the CCU 17. Video image data acquired by the camera head 13 and processed by the CCU 17 is converted to images, which can be displayed on a monitor 29, recorded by recorder 27, and/or used to generate static images, hard copies of which can be produced by printer 28.

FIG. 1B illustrates an open-field imaging device 60, which is another example of a type of imaging device that can be connected to an imaging controller via an, e.g. authenticable, cable, as discussed herein. Open-field imaging device 60 can be used as part of an imaging system, such as system 10 of FIG. 1A, for various purposes, including for visualizing blood flow in vessels and related tissue perfusion during plastic, microsurgical, reconstructive, and gastrointestinal procedures. As may be seen in FIG. 1B, the open-field imaging device 60 includes a control surface 62, a window frame 64 and a nosepiece 66. The open-field imaging device 60 is in this example connectable to the light source 14A via a light guide cable 15, through which the light is provided to the imaging field via ports in the window frame 64. The open-field imaging device 60 is connectable to the CCU 17 via an, e.g. authenticable, data cable 18, according to the principles described herein, which can transmit power, imaging data, and any other types of data.

The control surface 62 here includes focus buttons 63a (decreasing the working distance) and 63b (increasing the working distance) that control, e.g., outlet angles of the light beams for controlling a working distance at which the light beams substantially overlap for illuminating a target area. Other buttons on the control surface 62 may be programmable and may be used for various other functions, e.g., excitation laser power on/off, display mode selection, white light imaging white balance, saving a screenshot, and so forth. In some examples, the control surface functions can be communicated to the CCU 17 via non-imaging data communication lines in the cable 18, as discussed further below.

Existing Imaging Systems and their Deficiencies

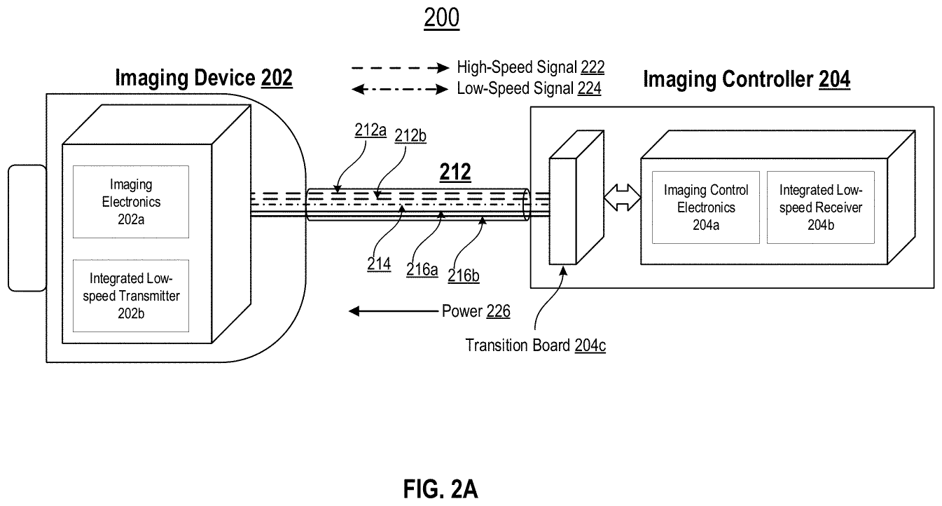

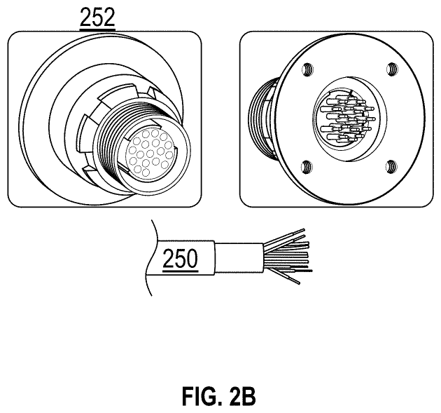

FIG. 2A illustrates an exemplary existing imaging system 200 that includes an imaging device 202 connected to an imaging controller 204 by a cable 212. The imaging device 202 comprises imaging electronics 202a (e.g., camera electronics) configured to generate image data. In some examples, the imaging device 202 is further configured to generate control signals, for example, based on user inputs to the imaging device 202 (e.g., via button presses of buttons on the imaging device). The imaging device 202 further comprises an integrated low-speed transmitter 202b for transmitting the generated image data and the generated control signals. In some examples, the transmitter 202b is configured to transmit data around or below 3 gigabits per second.

The imaging system 200 comprises an image controller 204. The imaging controller 204 comprises imaging control electronics 204a configured to generate control signals. The imaging controller 204 further comprises an integrated low-speed receiver 204b. In some examples, the receiver 204b is configured to receive data around or below 3 gigabits per second. In the depicted example, the imaging controller 204 further comprises a connector board 204c. The connector board 204c is configured to connect a cable connector to the connector interface of the imaging controller 204. In some examples, the connector board 204c does not enclose active electronic components.

The imaging system 200 comprises a cable 212 for connecting the imaging device 202 and the imaging controller 204. The cable transmits data between the imaging device 202 and the imaging controller 204. Specifically, the cable comprises multiple conductors. In the depicted example, the cable comprises five conductors or five conductive wires. The cable comprises two conductive wires 212a and 212b for transmitting high-speed signals 222 (e.g., image data generated by the imaging device) from the imaging device 202 to the imaging controller 204. The cable further comprises a conductive wire 214 for transmitting low-speed signals 224 (e.g., control signals) from the imaging device 202 to the imaging controller 204 and from the imaging controller 204 to the imaging device 202. The cable further comprises a conductive wire 216a for providing power from the imaging controller 204 to the imaging device 202 and a conductive wire 216b for providing ground for the power.

While the cable 212 of the imaging system 200 comprises 5 separate conductive wires, it should be appreciated that other examples of the multi-conductor cable 212 can comprise additional conductive wires. For example, if the imaging device comprises three sensors for generating red, green, and blue image data, the three sensors would independently generate three separate streams of image data. Accordingly, the cable may need to accommodate three separate conductive wires for transmitting the three streams of data (e.g., red, green, and blue image data), respectively, in a synchronized manner, in addition to conductive wires for transmitting control signals and power.

FIG. 2B illustrates an exemplary cable and an exemplary cable interface of an existing imaging system, in accordance with some examples. The cable 250 is another example of cable 212 in FIG. 2A. Instead of having 5 wires, the cable 250 has 18 conductive wires for transmitting image data, power, and control signals. Accordingly, the imaging controller comprises an interface 252 for connecting the 18 wires. As shown, a relatively large interface 252 needs to be provided for accommodating the 18 wires. As the data rate increases, an even larger number of conductive wires may be required to support the increasing data rate, thus increasing the size and complexity of the hardware components.

The imaging system 200 suffers from a number of deficiencies. First, because image data is transmitted via multiple conductive wires (e.g., 212a and 212b in FIG. 2A), the system needs to synchronize the multiple streams of data transmitted via multiple conductive wires. The synchronization may need to be performed on both the transmitter side and the receiver side. Synchronization can become more complex to implement as the number of conductive wires increases in the cable to support a higher data rate. Further, the multiple conductive wires in the same cable (e.g., 212a, 212b, 214, 216a, and 216b in FIG. 2A) can cause interferences among the transmitted signals and compromise the integrity of the signals. Further still, a cable having a specific number of conductive wires for separately transmitting the specific number of data streams can be difficult to integrate into a variety of imaging systems.

### Exemplary Imaging Systems Comprising Single-Conductor Cable(s)

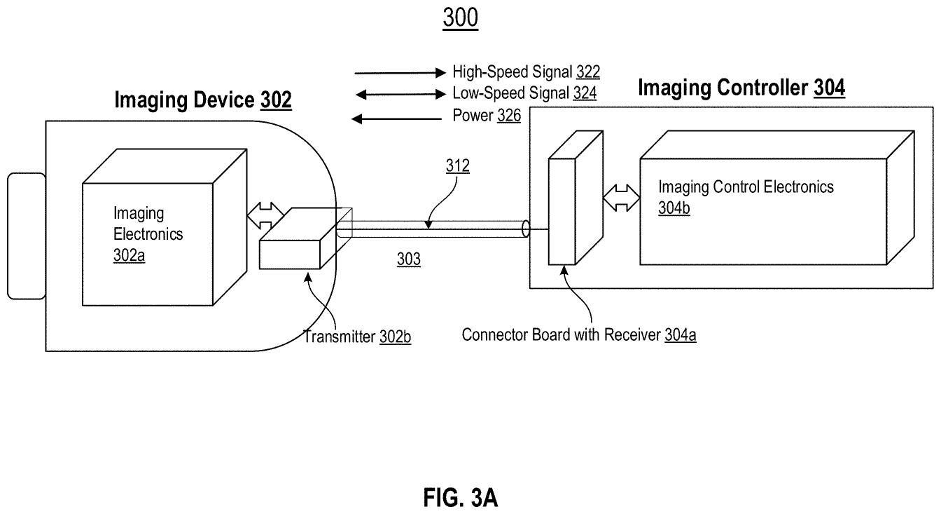

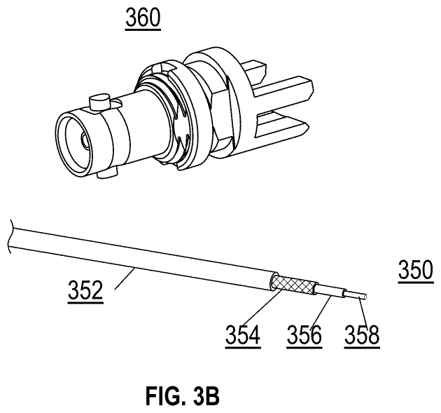

FIG. 3A illustrates an exemplary imaging system 300 (e.g., for acquiring surgical image data), according to some examples. The imaging system comprises an imaging device 302, an imaging controller 304, and a communication cable 303 for connecting the imaging device to the imaging controller, as described in detail below.

The imaging device 302 may be a medical imaging device or a non-medical imaging device. The imaging device may be a surgical imaging device. The imaging device may be any one of an endoscope camera head, a surgical microscope camera head, or an open field medical camera head. The imaging device 302 can be an endoscopic camera head 13 of FIG. 1A or open-field imaging device 60 of FIG. 1B. The imaging device 302 comprises imaging electronics 302a (e.g., camera electronics) configured to generate image data. In some examples, the imaging device 302 is further configured to generate control signals, for example, based on user inputs to the imaging device 302 (e.g., via button presses of buttons on the imaging device). The imaging device further comprises a transmitter 302b described in detail below.

The imaging controller 304 may be a controller that can be communicatively coupled with the imaging device 302 to receive image data from the imaging device 302 for processing and/or display and to control one or more imaging and/or non-imaging functions of the imaging device 302, such as CCU 17 of FIG. 1A. The imaging controller 304 comprises imaging control electronics 304b configured to generate control signals. In the depicted example, the imaging controller 304 further comprises a connector board. The connector board is configured to connect a cable connector to the connector interface of the imaging controller 304. The connector board houses a receiver 304a, described in detail below.

With reference to FIG. 3A, the cable 303 is configured to communicatively connect the imaging device 302 to the imaging controller 304. Cable 202 includes a distal end and a proximal end. A cable body extends between the proximal end and the distal end of the cable. The cable body houses a single conductor or a single conductive wire 312. This is in contrast to the cable 212 in the imaging system 200, which comprises five conductive wires.

The single conductive wire 312 is configured to perform three operations by itself: a first operation of transmitting high-speed signals 322 (e.g., surgical image data) from the imaging device 302 to the imaging controller 304, a second operation of transmitting low-speed signals 324 (e.g., non-image data such as control signals) between the imaging device 302 and the imaging controller 304, and a third operation of transmitting power 326 from the imaging controller 304 to the imaging device 302. In some examples, the three operations can be performed simultaneously by the single conductive wire 312.

With respect to the first operation of transmitting high-speed signals 322, the transmitted image data can comprise data acquired by one sensor or a plurality of sensors of the imaging device 302. The surgical image data can comprise at least one of pixel data and voxel data.

With respect to the second operation of transmitting low-speed signals 324, the transmitted control signals can comprise one or more first control signals generated by the imaging controller for controlling various parameters and features of the imaging device, such as shutter speed and gain. The one or more first control signals are transmitted from the imaging controller to the imaging device via the cable 303. The transmitted control signals can further comprise one or more second control signals generated by the imaging device 302. The imaging device 302 may include one or more non-imaging components, such as user interface components, such as buttons and switches, sensors, such as accelerometers and gyroscopes, displays, and controllers for controlling the non-imaging components. Thus, the one or more second control signals can be generated, for example, by user interface controls (e.g., zoom-in and zoom-out buttons) on the imaging device. The one or more second control signals are transmitted from the imaging device to the imaging controller via the cable 303.

In some examples, the cable 303 is a coaxial cable. FIG. 3B illustrates an exemplary cable and an exemplary cable interface, in accordance with some examples. The cable 350 (e.g., cable 303 in FIG. 3A) comprises an inner conductor 358 (i.e., conductive wire 312) surrounded by a dielectric layer 356, which is in turn surrounded by a cable shielding 354. In this example, the inner conductor is made of a conductive material, such as copper. In this example, the cable 350 further comprises a dielectric outer jacket 352. The single conductive wire 350 is configured to provide power (e.g., power 326 in FIG. 3A) from the imaging controller to the imaging device. The cable shielding 354 can be configured to provide ground to the power. As shown, the imaging controller comprises an interface 360 for connecting a single-conductor cable.

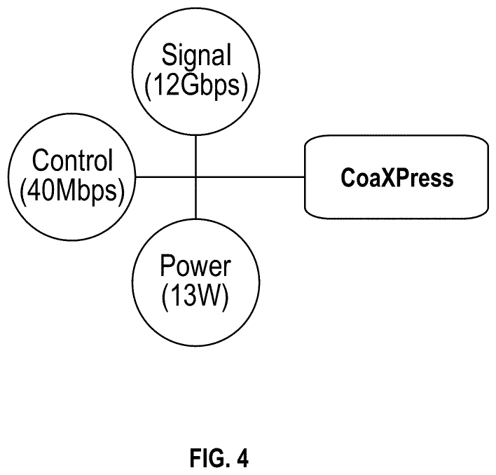

In some examples, the cable performs the first, second, and third operation according to the CoaXPress protocol. FIG. 4 illustrates exemplary single conductor data rates of the first, second, and third operation, including transmission of the image data (e.g., surgical image data) at up to around 12 gigabits per second. In alternative examples, the cable of the imaging system can be configured with additional conductors in order to transmit the image data (e.g., surgical image data) at over 12 gigabits per second, for example, 15 gigabits per second, 18 gigabits per second, 20 gigabits per second, 25 gigabits per second, 30 gigabits per second, 40 gigabits per second, 50 gigabits per second, 100 gigabits per second, 200 gigabits per second, 1 terabit per second, etc. In some examples, the cable of the imaging system is configured to transmit the control signal at around or over 40 megabits per second, for example, 45 megabits per second, 50 megabits per second, 100 megabits per second, etc. In some examples, the transmission rate of the control signals can scale or be adjusted based on the transmission rate of the image data.

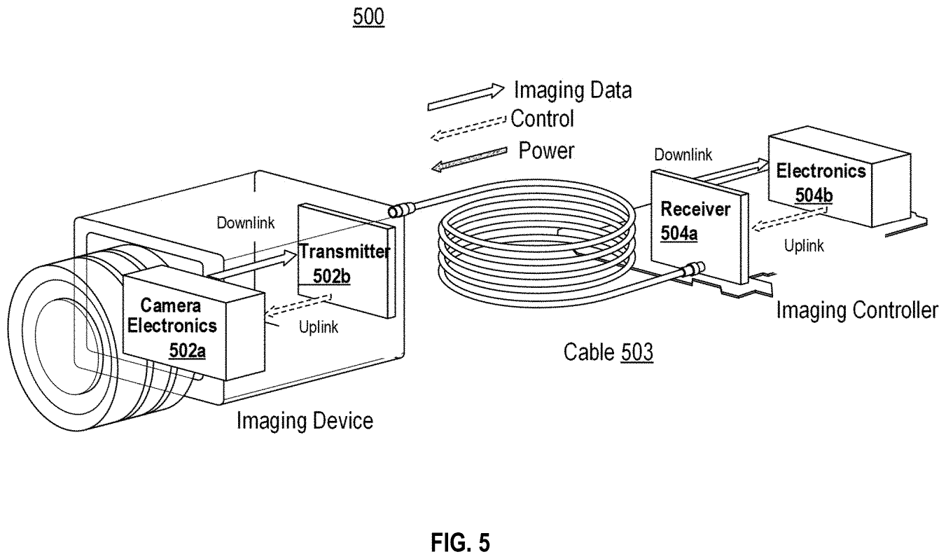

Turning back to FIG. 3A, the distal end of the communication cable 303 is connected to a transmitter 302b of the imaging device 302. Further, the proximal end of the communication cable is connected to a receiver 304a of the imaging controller 304. In this example, the receiver 304a is housed in the connector board of the imaging controller 304. The transmitter 302b can be configured to transmit the high-speed signals 322 (e.g., surgical image data) according to the CoaXPress protocol, and the receiver can be configured to receive the high-speed signals 322 (e.g., surgical image data) according to the CoaXPress protocol. The transmitter and the receiver are described further with reference to FIG. 5 below.

FIG. 5 illustrates an exemplary imaging system 500 operating according to the CoaXPress protocol, in accordance with some examples. In some examples, the imaging system 500 is the imaging system 300 in FIG. 3A. The camera electronics 502a (e.g., one or more sensors) generates image data, which is provided to the transmitter 502b (e.g., transmitter 302b in FIG. 3A) for transmission. In some examples, the transmitter reformats (e.g., serializes) the image data into a different format before transmission. The transmitter 502b is configured to transmit image data using a downlink (e.g., around or over 12 gigabits per second) via the cable 503. The transmitter can further be configured to receive control signals using an uplink (e.g., around or over 40 megabits per second). In such case, the transmitter 502b can be referred to as a transceiver. In some examples, the transmitter provides a clock and a data recovery mechanism for the acquired data from the camera electronics 502a.

The cable 503 (e.g., cable 303 in FIG. 3A) can be a coaxial cable for transmitting the image data from the transmitter 502b to the receiver 504a of the imaging controller. The cable further transmits control signals from the receiver 504a to the transmitter 502b, as well as from the transmitter 502b to the receiver 504a. The cable further provides power from the receiver 504a to the transmitter 502b. In some examples, the cable is around or below 40 meters in length.

The transmitted image data is received at the receiver 504b of the imaging controller. The receiver can further provide the received image data to the imaging control electronics 504b. The receiver 504b is configured to receive image data using a downlink (e.g., around or over 12 gigabits per second) via the cable 503. The receiver can further be configured to transmit control signals using an uplink (e.g., around or over 40 megabits per second). In such case, the receiver 504b can be referred to as a transceiver. In some examples, the receiver reformats the received image data. In some examples, the receiver is configured to perform equalization on the received image data and the control signals. Equalization is configured to recover data loss. In some examples, equalization reverses distortion incurred by a signal transmitted through a channel.

Turning back to FIG. 3A, in an example of operation, the imaging controller 304 is energized first. The receiver 304a can have DC bias, which enables the system to transmit power over the single conductor. Once the receiver 304a is powered on, it supplies the DC power to the transmitter 302b connected through the cable 303. Once the transmitter 302b is energized, the imaging electronics 302a are energized. A control communication is established from the receiver to the transmitter. The transmitter 302b is incorporated in the imaging device (e.g., camera head) and interfaces with the imaging electronics 302a. The imaging device 302 transmits the generated digital image data to the receiver 304a over the same cable 303.

The imaging device 302 can accommodate any number of sensors. The image data generated by the sensors can be all transmitted via the single-conductor cable 303 to the imaging controller 304. In some examples, the cable has a maximum data transmission rate (e.g., about 13 gigabits per second). If the data generated by the imaging device 302 exceeds the maximum rate (e.g., due to the number of sensors), additional cables can be added to the imaging system 300 to support the data rate. For example, in addition to cable 303, the imaging system 300 can further comprise a second single-connector cable for connecting the imaging device 302 to the imaging controller 304. The second cable is configured to transmit a second set of image data from the imaging device to the imaging controller. In some examples, the second cable is connected to the same transmitter 302b and receiver 304a. In some examples, only one of the two cables is used to provide power to the imaging device.

In some examples, the cable 303 is an optical cable configured to transmit data between the imaging device 302 and the imaging controller 304 according to the CoaXPress protocol and a separate cable is used to transmit power to the imaging device 302. In some examples, the imaging system transmits data according to a different data transmission protocol, such as camera link, USB, Ethernet, analog, etc.

The distal end of the cable 303 can include a distal end connector for connecting the cable 303 to the imaging device 302. The proximal end of cable 303 can include a proximal end connector for connecting the cable to the imaging controller 304. The distal end connector and the proximal end connector can each be any suitable connector and may include any suitable number of contacts (e.g., pins and receptacles) for connecting one or more conductive wires. Suitable connectors may include one or more locking features for preventing or discouraging an end-user from disconnecting the connector. Examples of suitable locking features are a locking lever, a locking screw, a locking toggle, a locking nut, or a locking bayonet. In some examples, the locking feature is configured to require a tool for unlocking. Suitable connectors may also be dis-connectable connectors that enable an end-user to disconnect the connector in the field.

In some examples, the distal end connector, which is configured to connect the cable to the imaging device 302, may be configured for permanent or at least semi-permanent attachment to the imaging device 302 for preventing (or discouraging) an end-user from detaching the distal end connector from the imaging device 302. The distal end connector may include one or more locking features that prevent tool-less detachment of the distal end connector from the imaging device. The imaging device 302 and the distal end connector may e.g. be permanently or semi-permanently connected at the manufacturing facility and shipped to the end user as an assembled set. The distal end connector can include a sealing feature, such as an O-ring or grommet, for sealing the connection to the imaging device 302. This can protect the electronics within the imaging device 302 and/or cable 303 during sterilization.

In some examples, the proximal end connector of the cable 303 is configured for dis-connectable attachment to the imaging controller 304. As such, a user may be able to repeatedly disconnect the proximal end connector, such as by hand. Once disconnected, the imaging device 302 and attached cable 303 can be cleaned, sterilized, stored, used with a different imaging controller, repaired, or otherwise disposed of separately from the imaging controller 304.

The imaging system 300 provides many technical improvements over existing imaging systems (e.g., imaging system 200). The cable 303 of the imaging system 300 can rely on a single conductor or single conductive wire for transmitting image data (e.g., at around or over 12 gigabits per second), non-image data such as control signals (e.g., at around or over 40 megabits per second), and power. The single-conductor cable (e.g., coaxial cable) does not suffer from interferences that would occur in a multi-conductor cable (e.g., cable 212 in FIG. 2A). Further, the single-conductor cable does not require synchronization among multiple data streams, thus resulting in a simplified hardware and software assembly. The single-conductor cable 303 provides a simplified interface (e.g., FIG. 3B) that is easier to manufacture, to use, and to maintain. The single-conductor cable is smaller in diameter and more pliable, making it less susceptible to wear and tear and to environmental factors (e.g., moisture, sterilization). The single-conductor cable also requires less material and is longer lasting and more environmentally friendly. Further, the single-conductor cable requires only one termination compared to multi-conductor cable requiring multiple terminations. Standard industrial connectors can be used to terminate the single wire coaxial cable, and these standard connectors further can be customized mechanically to fit cable design needs easily. Printed circuit boards (PCBs) built to mount these customized connectors and terminate these customized connectors can be designed freely.

The combination of hardware and software in the imaging system 300 allows any number of image sensors to be integrated without changes to the cable assembly, and further allows the transfer of data without dedicated channels. The transmitter 302b and the receiver 304a of the imaging system can e.g. be constructed from off-the-shelf components supporting the CoaXPress protocol, thus reducing manufacturing cost. The transmitter and the receiver can be fully customizable, thus providing a flexible platform for software and hardware development.

The imaging system 300 also provides a number of mechanical advantages. Endoscopic medical cameras require sterilization after each use. The sterilization process, which can involve autoclave or hydrogen peroxide gas, can be very harsh to the typical cable jacket material (e.g., PVC or polyurethane). Silicone cable jacket is a superior material to withstand repeated sterilization cycles. However, silicone rubber has lower mechanical strength than PVC and polyurethane. Thus, the silicone jacket is typically designed to have a larger diameter. The single-conductor system 300 significantly reduces the number of the cable conductors, in some cases from 18 conductors (e.g., FIG. 2B) to 1 conductor (e.g., FIG. 3B). Thus, in system 300, a silicone jacket with a smaller diameter can be used, resulting in a small and ergonomic medical camera cable that can also withstand repeated sterilization. For example, a 3-meter multi-conductor cable can be 0.275″ in diameter, while a 3-meter single-conductor cable can be 0.200″ in diameter, resulting in a 66% reduction in size (i.e., cross-sectional area). As another example, a 10-meter multi-conductor cable can be 0.375″ in diameter, while a 10-meter single-conductor cable can be 0.160″ in diameter, resulting in a 82% reduction in size. Further, a medical camera cable can be subjected to various bending and flexing during use, a smaller cable with less wires will significantly improve the flex and bending life of the camera cable.

Further, in the existing multi-conductor imaging system 200, twinaxial cables may be used for high-speed data transmission but have many limitations. For example, to transmit 12 gigabits per second, four twinaxial wires may be needed. In contrast, the imaging system 300 uses one coaxial wire to support the same data rate. If the data rate increases to 24 gigabits per second, a twinaxial cable design would require 8 twinaxial wires, whereas the system 300 would only require 2 coaxial wires (e.g., in two single-conductor cables respectively). Further, endoscopy camera is preferred to be small and compact for ergonomic purposes. The transmitter is placed in the camera housing and the transmitter chip is compact (e.g., around 4×4 mm) and does not cause the size of the camera housing to increase. Thus, the imaging system 300 results a much smaller and ergonomic cable that is able to withstand sterilization. Thus, the imaging system 300 further provides a compact solution.

### Example System for Use in Generating Imaging Data

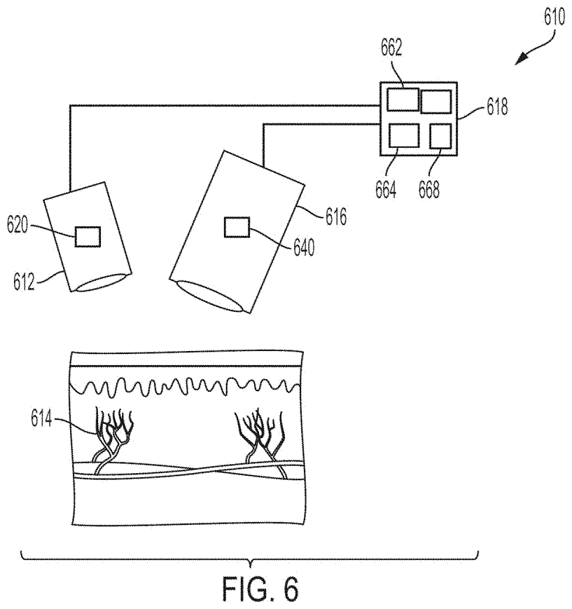

A system for collecting medical imaging data, such as system 10 of FIG. 1A, may include one or more imaging systems for acquiring a time series of images of tissue (e.g., a time series of fluorescence images, a time series of white light images, etc.). In some examples, an imaging system is a fluorescence imaging system. FIG. 6 is a schematic example of a fluorescence imaging system 610, according to some examples. The fluorescence imaging system 610 comprises a light source 612 to illuminate the tissue of the subject to induce fluorescence emission from a fluorescence imaging agent 614 in the tissue of the subject (e.g., in blood, in urine, in lymph fluid, in spinal fluid or other body fluids or tissues), an image acquisition assembly 616 arranged for generating the time series and/or the subject time series of fluorescence images from the fluorescence emission, and a processor assembly 618 arranged for processing the generated time series/subject time series of fluorescence images. The processor assembly 618 may include memory 668 with instructions thereon, a processor module 662 arranged for executing the instructions on memory 668 to process the time series and/or subject time series of fluorescence images, and a data storage module 664 to store the unprocessed and/or processed time series and/or subject time series of fluorescence images. In some variations, the memory 668 and data storage module 664 may be embodied in the same storage medium, while in other variations the memory 668 and the data storage module 664 may be embodied in different storage mediums. The system 610 may further include a communication module 666 for transmitting images and other data, such as some or all of the time series/subject time series of fluorescence images or other input data, spatial maps, subject spatial maps, and/or a tissue numerical value (quantifier), to an imaging data processing hub.

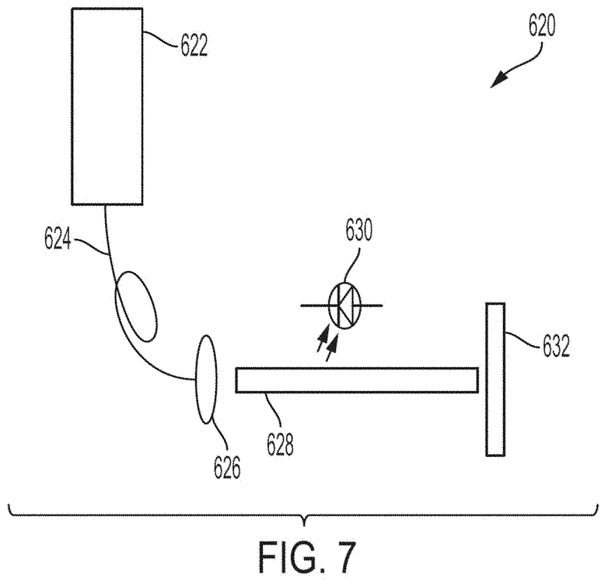

In this example, the light source 612 includes an illumination module 620. Illumination module 620 may include a fluorescence excitation source arranged for generating an excitation light having a suitable intensity and a suitable wavelength for exciting the fluorescence imaging agent 614. As shown in FIG. 7, the illumination module 620 may comprise a laser diode 622 (e.g., which may comprise, for example, one or more fiber-coupled diode lasers) arranged for providing an excitation light to excite the fluorescence imaging agent (not shown) in tissue of the subject. Examples of other sources of the excitation light which may be used in various examples include one or more LEDs, arc lamps, or other illuminant technologies of sufficient intensity and appropriate wavelength to excite the fluorescence imaging agent in the tissue. For example, excitation of the fluorescence imaging agent in blood, wherein the fluorescence imaging agent is a fluorescence dye with near infra-red excitation and emission characteristics, may be performed using one or more 793 nm, conduction-cooled, single bar, fiber-coupled laser diode modules from DILAS Diode Laser Co, Germany.

The light output from the light source 612 may be projected through one or more optical elements to shape and guide the output being used to illuminate the tissue area of interest. The optical elements may include one or more lenses, light guides, and/or diffractive elements so as to ensure a flat field over substantially the entire field of view of the image acquisition assembly 616. The fluorescence excitation source may be selected to emit at a wavelength close to the absorption maximum of the fluorescence imaging agent 614 (e.g., indocyanine green (ICG), etc.). For example, as shown in FIG. 7, the output 624 from the laser diode 622 may be passed through one or more focusing lenses 626, and then through a homogenizing light pipe 628 such as, for example, light pipes commonly available from Newport Corporation, USA. Finally, the light may be passed through an optical diffractive element 632 (e.g., one or more optical diffusers) such as, for example, ground glass diffractive elements also available from Newport Corporation, USA. Power to the laser diode 622 may be provided by, for example, a high-current laser driver such as those available from Lumina Power Inc. USA. The laser may optionally be operated in a pulsed mode during the image acquisition process. An optical sensor such as a solid state photodiode 630 may be incorporated into the illumination module 620 and may sample the illumination intensity produced by the illumination module 620 via scattered or diffuse reflections from the various optical elements. In some variations, additional illumination sources may be used to provide guidance when aligning and positioning the module over the area of interest.

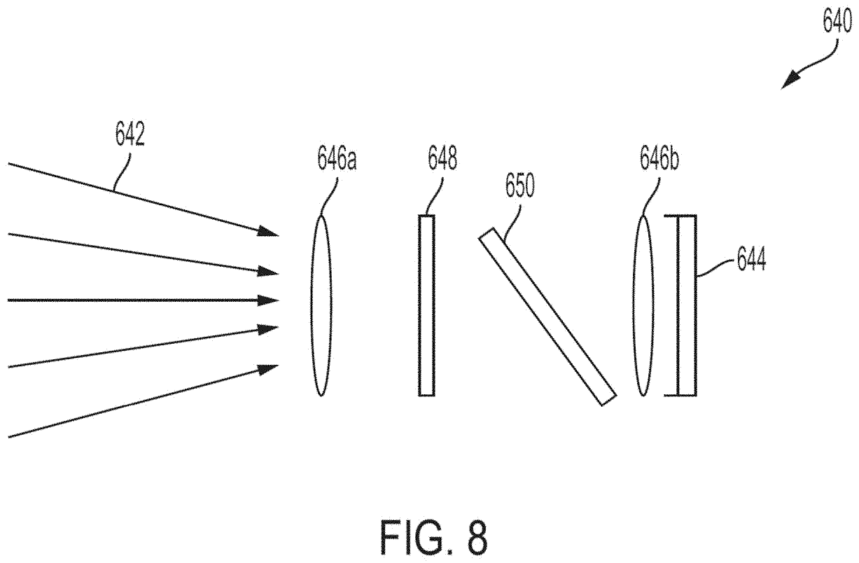

Referring again to FIG. 6, in this example, the image acquisition assembly 616 is be a component of a fluorescence imaging system 610 configured to acquire the time series and/or subject time series of fluorescence images from the fluorescence emission from the fluorescence imaging agent 614. The image acquisition assembly 616 may include a camera module 640, which may include an imaging device, such as endoscopic camera 13 of FIG. 1A, open-field imaging device 60 of FIG. 1B, and imaging device 201 of FIG. 2A and FIG. 2B, connected to an imaging controller, such as imaging controller 203, via a cable, here an authenticable cable with memory, such as cable 202. As shown in FIG. 8, the camera module 640 may acquire images of the fluorescence emission 642 from the fluorescence imaging agent in the tissue by using a system of imaging optics (e.g., 646a, 646b, 648 and 650) to collect and focus the fluorescence emission onto an image sensor assembly 644. The image sensor assembly 644 may comprise at least one 2D solid state image sensor. The solid state image sensor may be a charge coupled device (CCD), a CMOS sensor, a CID or similar 2D sensor technology. The charge that results from the optical signal transduced by the image sensor assembly 644 is converted to an electrical video signal, which includes both digital and analog video signals, by the appropriate read-out and amplification electronics in the camera module 640.

According to an exemplary variation of a fluorescent imaging system, the light source may provide an excitation wavelength of about 800 nm+/−10 nm, and the image acquisition assembly uses emission wavelengths of >820 nm with NIR-compatible optics for, for example, ICG fluorescence imaging. In an exemplary example, the NIR-compatible optics may include a CCD monochrome image sensor having a GigE standard interface and a lens that is compatible with the sensor with respect to optical format and mount format (e.g., C/CS mount).

The processor module 662 may comprise any computer or computing means such as, for example, a tablet, laptop, desktop, networked computer, or dedicated standalone microprocessor. For instance, the processor module 662 may include one or more central processing units (CPU). In an exemplary example, the processor module 662 is a quad-core, 2.5 GHz processor with four CPUs where each CPU is a microprocessor such as a 64-bit microprocessor (e.g., marketed as INTEL Core i3, i5, or i7, or in the AMD Core FX series). However, in other examples, the processor module 662 may be any suitable processor with any suitable number of CPUs and/or other suitable clock speed.

Inputs for the processor module 662 may be taken, for example, from the image sensor 644 of the camera module 640 shown in FIG. 8, from the solid state photodiode 630 in the illumination module 620 in FIG. 7, and/or from any external control hardware such as a footswitch or remote-control. Output is provided to the laser diode driver and optical alignment aids. As shown in the example of FIG. 6, the processor assembly 618 may have a data storage module 664 with the capability to save the time series/subject time series of images, or data representative thereof, or other input data to a tangible non-transitory computer readable medium such as, for example, internal memory (e.g. a hard disk or flash memory), so as to enable recording and processing of acquired data. In some variations, the processor module 662 may have an internal clock to enable control of the various elements and ensure correct timing of illumination and sensor shutters. In some variations, the processor module 662 may also provide user input and graphical display of outputs. The fluorescence imaging system may optionally be configured with a communication unit 666, such as a wired or wireless network connection or video output connection for transmitting the time series of fluorescence images as they are being acquired or played back after recording. The communication unit 666 may additionally or alternatively transmit processed data, such as a spatial map, a subject spatial map, and/or tissue numerical value.

In operation of the exemplary system described in FIGS. 6-8, the subject is positioned relative to fluorescence imaging system 610 such that an area of interest (e.g., target tissue region) is located beneath the light source 612 and the image acquisition assembly 616 such that the illumination module 620 of light source 612 produces a substantially uniform field of illumination across substantially the entire area of interest. In some variations, prior to the administration of the fluorescence imaging agent 614 to the subject, an image may be acquired of the area of interest for the purposes of background deduction. To acquire fluorescence images/subject fluorescence images, the operator of the fluorescence imaging system 610 may initiate the acquisition of the time series/subject time series of fluorescence images by depressing a remote switch or foot-control, or via a keyboard (not shown) connected to the processor assembly 618. As a result, the light source 612 is turned on and the processor assembly 618 begins recording the fluorescence image data/subject fluorescence image data provided by the image acquisition assembly 616. When operating in the pulsed mode of the example, the image sensor 644 in the camera module 640 is synchronized to collect fluorescence emission following the laser pulse produced by the diode laser 622 in the illumination module 620. In this way, maximum fluorescence emission intensity is recorded, and signal-to-noise ratio is optimized. In this example, the fluorescence imaging agent 614 is administered to the subject and delivered to the area of interest via arterial flow. Acquisition of the time series/subject time series of fluorescence images is initiated, for example, shortly after administration of the fluorescence imaging agent 614, and the time series of fluorescence images from substantially the entire area of interest is acquired throughout the ingress of the fluorescence imaging agent 614. The fluorescence emission from the region of interest is collected by the collection optics of the camera module 640. Residual ambient and reflected excitation light is attenuated by subsequent optical elements (e.g., optical element 650 in FIG. 8 which may be a filter) in the camera module 640 so that the fluorescence emission can be acquired by the image sensor assembly 644 with minimal interference by light from other sources.

In some variations, following the acquisition or generation of the time series/subject time series of fluorescence images, the processor assembly 618 (e.g., processor module 662 or other processor) may then be initiated to execute instructions stored on memory 668 and process the imaging data before transmission to the imaging data processing system. The system 610 may transmit, via connection 666, the spatial map/subject spatial map and/or any clinical correlations or diagnosis derived therefrom or both for display to the user in a composite display feed as, for example, a grayscale or false color image, and/or stored for subsequent use.

A computer program product, such as a tangible non-transitory computer readable medium having computer-executable (readable) program code embedded thereon, may provide instructions for causing one or more processors to, when executing the instructions, perform one or more of the methods described herein. Program code can be written in any appropriate programming language and delivered to the processor in many forms, including, for example, but not limited to information permanently stored on non-writeable storage media (e.g., read-only memory devices such as ROMs, CD-ROM disks, etc.), information alterably stored on writeable storage media (e.g., hard drives or the like), information conveyed to the processor through communication media, such as a local area network, a public network such as the Internet, or any type of media suitable for storing electronic instruction. When carrying computer readable instructions that implement the various examples of the methods described herein, such computer readable media represent examples of various examples. In various examples, the tangible non-transitory computer readable medium comprises all computer-readable media, and the present invention scope is limited to computer readable media wherein the media is both tangible and non-transitory.

A kit may include any part of the systems described herein and the fluorescence imaging agent such as, for example, a fluorescence dye such as ICG or any suitable fluorescence imaging agent. In further aspects, a kit may include a tangible non-transitory computer readable medium having computer-executable (readable) program code embedded thereon that may provide instructions for causing one or more processors, when executing the instructions, to perform one or more of the methods for characterizing tissue and/or predicting clinical data described herein. The kit may include instructions for use of at least some of its components (e.g., for using the fluorescence imaging agent, for installing the computer-executable (readable) program code with instructions embedded thereon, etc.). In yet further aspects, there is provided a fluorescence imaging agent such as, for example, a fluorescence dye for use in in the methods and systems described herein. In further variations, a kit may include any part of or the entire system described herein and a fluorescence agent such as, for example, a fluorescence dye such as ICG, or any other suitable fluorescence agent, or a combination of fluorescence agents.

### Example Imaging Agents for Use in Generating Imaging Data

According to some examples, in fluorescence medical imaging applications, the imaging agent is a fluorescence imaging agent such as, for example, ICG dye. The fluorescence imaging agent, such as ICG, may be pre-administered to the subject, prior to performing the measurement of signal intensity arising from the fluorescence imaging agent. ICG, when administered to the subject, binds with blood proteins and circulates with the blood in the tissue. The fluorescence imaging agent (e.g., ICG) may be administered to the subject as a bolus injection (e.g., into a vein or an artery) in a concentration suitable for imaging such that the bolus circulates in the vasculature and traverses the microvasculature. In other examples in which multiple fluorescence imaging agents are used, such agents may be administered simultaneously, e.g. in a single bolus, or sequentially in separate boluses. The fluorescence imaging agents may be pre-administered to the subject, prior to performing the measurement of signal intensity arising from the fluorescence imaging agent. In some examples, the fluorescence imaging agent may be administered by a catheter. In certain examples, the fluorescence imaging agent may be administered less than an hour in advance of performing the measurement of signal intensity arising from the fluorescence imaging agent. For example, the fluorescence imaging agent may be administered to the subject less than 30 minutes in advance of the measurement. In yet other examples, the fluorescence imaging agent may be administered at least 30 seconds in advance of performing the measurement. In still other examples, the fluorescence imaging agent may be administered contemporaneously with performing the measurement.

According to some examples, the fluorescence imaging agent may be administered in various concentrations to achieve a desired circulating concentration in the blood. For example, in examples where the fluorescence imaging agent is ICG, it may be administered at a concentration of about 2.5 mg/mL to achieve a circulating concentration of about 5 μM to about 10 μM in blood. In various examples, the upper concentration limit for the administration of the fluorescence imaging agent is the concentration at which the fluorescence imaging agent becomes clinically toxic in circulating blood, and the lower concentration limit is the instrumental limit for acquiring the signal intensity data arising from the fluorescence imaging agent circulating with blood to detect the fluorescence imaging agent. In various other examples, the upper concentration limit for the administration of the fluorescence imaging agent is the concentration at which the fluorescence imaging agent becomes self-quenching. For example, the circulating concentration of ICG may range from about 2 μM to about 10 mM. Thus, in one aspect, the method comprises the step of administration of the imaging agent (e.g., a fluorescence imaging agent) to the subject and acquisition of the signal intensity data (e.g., video) prior to processing the signal intensity data according to the various examples. In another aspect, the method excludes any step of administering the imaging agent to the subject.

According to some examples, a suitable fluorescence imaging agent for use in fluorescence imaging applications to generate fluorescence image data is an imaging agent which can circulate with the blood (e.g., a fluorescence dye which can circulate with, for example, a component of the blood such as lipoproteins or serum plasma in the blood) and transit vasculature of the tissue (i.e., large vessels and microvasculature), and from which a signal intensity arises when the imaging agent is exposed to appropriate light energy (e.g., excitation light energy, or absorption light energy). In various examples, the fluorescence imaging agent comprises a fluorescence dye, an analogue thereof, a derivative thereof, or a combination of these. A fluorescence dye includes any non-toxic fluorescence dye. In certain examples, the fluorescence dye optimally emits fluorescence in the near-infrared spectrum. In certain examples, the fluorescence dye is or comprises a tricarbocyanine dye. In certain examples, the fluorescence dye is or comprises ICG, methylene blue, or a combination thereof. In other examples, the fluorescence dye is or comprises fluorescein isothiocyanate, rhodamine, phycoerythrin, phycocyanin, allophycocyanin, o-phthaldehyde, fluorescamine, rose Bengal, trypan blue, fluoro-gold, or a combination thereof, excitable using excitation light wavelengths appropriate to each dye. In some examples, an analogue or a derivative of the fluorescence dye may be used. For example, a fluorescence dye analog or a derivative includes a fluorescence dye that has been chemically modified, but still retains its ability to fluoresce when exposed to light energy of an appropriate wavelength.

In various examples, the fluorescence imaging agent may be provided as a lyophilized powder, solid, or liquid. In certain examples, the fluorescence imaging agent may be provided in a vial (e.g., a sterile vial), which may permit reconstitution to a suitable concentration by administering a sterile fluid with a sterile syringe. Reconstitution may be performed using any appropriate carrier or diluent. For example, the fluorescence imaging agent may be reconstituted with an aqueous diluent immediately before administration. In various examples, any diluent or carrier which will maintain the fluorescence imaging agent in solution may be used. As an example, ICG may be reconstituted with water. In some examples, once the fluorescence imaging agent is reconstituted, it may be mixed with additional diluents and carriers. In some examples, the fluorescence imaging agent may be conjugated to another molecule, such as a protein, a peptide, an amino acid, a synthetic polymer, or a sugar, for example to enhance solubility, stability, imaging properties, or a combination thereof. Additional buffering agents may optionally be added including Tris, HCl, NaOH, phosphate buffer, and/or HEPES.

A person of skill in the art will appreciate that, although a fluorescence imaging agent was described above in detail, other imaging agents may be used in connection with the systems, methods, and techniques described herein, depending on the optical imaging modality. Such fluorescence agents may be administered into body fluid (e.g., lymph fluid, spinal fluid) or body tissue.

In some variations, the fluorescence imaging agent used in combination with the methods, systems and kits described herein may be used for blood flow imaging, tissue perfusion imaging, lymphatic imaging, or a combination thereof, which may performed prior to, during or after an invasive surgical procedure, a minimally invasive surgical procedure, a non-invasive surgical procedure, or a combination thereof. The method of blood flow imaging, tissue perfusion imaging, lymphatic imaging, or a combination thereof, per se may exclude any invasive surgical step. Examples of invasive surgical procedure which may involve blood flow and tissue perfusion include a cardiac-related surgical procedure (e.g., CABG on pump or off pump) or a reconstructive surgical procedure. An example of a non-invasive or minimally invasive procedure includes wound (e.g., chronic wound such as for example pressure ulcers) treatment and/or management. In this regard, for example, a change in the wound over time, such as a change in wound dimensions (e.g., diameter, area), or a change in tissue perfusion in the wound and/or around the peri-wound, may be tracked over time with the application of the methods and systems. Examples of lymphatic imaging include identification of one or more lymph nodes, lymph node drainage, lymphatic mapping, or a combination thereof. In some variations such lymphatic imaging may relate to the female reproductive system (e.g., uterus, cervix, vulva).

In variations relating to cardiac applications, the imaging agent(s) (e.g., ICG alone or in combination with another imaging agent) may be injected intravenously through, for example, the central venous line, bypass pump and/or cardioplegia line to flow and/or perfuse the coronary vasculature, microvasculature and/or grafts. ICG may be administered as a dilute ICG/blood/saline solution down the grafted vessel such that the final concentration of ICG in the coronary artery is approximately the same or lower as would result from injection of about 2.5 mg (i.e., 1 ml of 2.5 mg/ml) into the central line or the bypass pump. The ICG may be prepared by dissolving, for example, 25 mg of the solid in 10 ml sterile aqueous solvent, which may be provided with the ICG by the manufacturer. One milliliter of the ICG solution may be mixed with 500 ml of sterile saline (e.g., by injecting 1 ml of ICG into a 500 ml bag of saline). Thirty milliliters of the dilute ICG/saline solution may be added to 10 ml of the subject's blood, which may be obtained in an aseptic manner from the central arterial line or the bypass pump. ICG in blood binds to plasma proteins and facilitates preventing leakage out of the blood vessels. Mixing of ICG with blood may be performed using standard sterile techniques within the sterile surgical field. Ten ml of the ICG/saline/blood mixture may be administered for each graft. Rather than administering ICG by injection through the wall of the graft using a needle, ICG may be administered by means of a syringe attached to the (open) proximal end of the graft. When the graft is harvested surgeons routinely attach an adaptor to the proximal end of the graft so that they can attach a saline filled syringe, seal off the distal end of the graft and inject saline down the graft, pressurizing the graft and thus assessing the integrity of the conduit (with respect to leaks, side branches etc.) prior to performing the first anastomosis. In other variations, the methods, dosages or a combination thereof as described herein in connection with cardiac imaging may be used in any vascular and/or tissue perfusion imaging applications.

Lymphatic mapping is an important part of effective surgical staging for cancers that spread through the lymphatic system (e.g., breast, gastric, gynecological cancers). Excision of multiple nodes from a particular node basin can lead to serious complications, including acute or chronic lymphedema, paresthesia, and/or seroma formation, when in fact, if the sentinel node is negative for metastasis, the surrounding nodes will most likely also be negative. Identification of the tumor draining lymph nodes (LN) has become an important step for staging cancers that spread through the lymphatic system in breast cancer surgery for example. LN mapping involves the use of dyes and/or radiotracers to identify the LNs either for biopsy or resection and subsequent pathological assessment for metastasis. The goal of lymphadenectomy at the time of surgical staging is to identify and remove the LNs that are at high risk for local spread of the cancer. Sentinel lymph node (SLN) mapping has emerged as an effective surgical strategy in the treatment of breast cancer. It is generally based on the concept that metastasis (spread of cancer to the axillary LNs), if present, should be located in the SLN, which is defined in the art as the first LN or group of nodes to which cancer cells are most likely to spread from a primary tumor. If the SLN is negative for metastasis, then the surrounding secondary and tertiary LN should also be negative. The primary benefit of SLN mapping is to reduce the number of subjects who receive traditional partial or complete lymphadenectomy and thus reduce the number of subjects who suffer from the associated morbidities such as lymphedema and lymphocysts.

The current standard of care for SLN mapping involves injection of a tracer that identifies the lymphatic drainage pathway from the primary tumor. The tracers used may be radioisotopes (e.g. Technetium-99 or Tc-99m) for intraoperative localization with a gamma probe. The radioactive tracer technique (known as scintigraphy) is limited to hospitals with access to radioisotopes require involvement of a nuclear physician and does not provide real-time visual guidance. A colored dye, isosulfan blue, has also been used, however this dye cannot be seen through skin and fatty tissue. In addition, blue staining results in tattooing of the breast lasting several months, skin necrosis can occur with subdermal injections, and allergic reactions with rare anaphylaxis have also been reported. Severe anaphylactic reactions have occurred after injection of isosulfan blue (approximately 2% of patients). Manifestations include respiratory distress, shock, angioedema, urticarial and pruritus. Reactions are more likely to occur in subjects with a history of bronchial asthma, or subjects with allergies or drug reactions to triphenylmethane dyes. Isosulfan blue is known to interfere with measurements of oxygen saturation by pulse oximetry and methemoglobin by gas analyzer. The use of isosulfan blue may result in transient or long-term (tattooing) blue coloration.

In contrast, fluorescence imaging in accordance with the various examples for use in SLN visualization, mapping, facilitates direct real-time visual identification of a LN and/or the afferent lymphatic channel intraoperatively, facilitates high-resolution optical guidance in real-time through skin and fatty tissue, visualization of blood flow, tissue perfusion or a combination thereof.

In some variations, visualization, classification or both of lymph nodes during fluorescence imaging may be based on imaging of one or more imaging agents, which may be further based on visualization and/or classification with a gamma probe (e.g., Technetium Tc-99m is a clear, colorless aqueous solution and is typically injected into the periareolar area as per standard care), another conventionally used colored imaging agent (isosulfan blue), and/or other assessment such as, for example, histology. The breast of a subject may be injected, for example, twice with about 1% isosulfan blue (for comparison purposes) and twice with an ICG solution having a concentration of about 2.5 mg/ml. The injection of isosulfan blue may precede the injection of ICG or vice versa. For example, using a TB syringe and a 30 G needle, the subject under anesthesia may be injected with 0.4 ml (0.2 ml at each site) of isosulfan blue in the periareolar area of the breast. For the right breast, the subject may be injected at 12 and 9 o'clock positions and for the left breast at 12 and 3 o'clock positions. The total dose of intradermal injection of isosulfan blue into each breast may be about 4.0 mg (0.4 ml of 1% solution: 10 mg/ml). In another exemplary variation, the subject may receive an ICG injection first followed by isosulfan blue (for comparison). One 25 mg vial of ICG may be reconstituted with 10 ml sterile water for injection to yield a 2.5 mg/ml solution immediately prior to ICG administration. Using a TB syringe and a 30G needle, for example, the subject may be injected with about 0.1 ml of ICG (0.05 ml at each site) in the periareolar area of the breast (for the right breast, the injection may be performed at 12 and 9 o'clock positions and for the left breast at 12 and 3 o'clock positions). The total dose of intradermal injection of ICG into each breast may be about 0.25 mg (0.1 ml of 2.5 mg/ml solution) per breast. ICG may be injected, for example, at a rate of 5 to 10 seconds per injection. When ICG is injected intradermally, the protein binding properties of ICG cause it to be rapidly taken up by the lymph and moved through the conducting vessels to the LN. In some variations, the ICG may be provided in the form of a sterile lyophilized powder containing 25 mg ICG with no more than 5% sodium iodide. The ICG may be packaged with aqueous solvent consisting of sterile water for injection, which is used to reconstitute the ICG. In some variations the ICG dose (mg) in breast cancer sentinel lymphatic mapping may range from about 0.5 mg to about 10 mg depending on the route of administration. In some variations, the ICG does may be about 0.6 mg to about 0.75 mg, about 0.75 mg to about 5 mg, about 5 mg to about 10 mg. The route of administration may be for example subdermal, intradermal (e.g., into the periareolar region), subareolar, skin overlaying the tumor, intradermal in the areola closest to tumor, subdermal into areola, intradermal above the tumor, periareolar over the whole breast, or a combination thereof. The injections may be prior to visualization and/or classification. The NIR fluorescent positive LNs (e.g., using ICG) may be represented as a black and white NIR fluorescence image(s) for example and/or as a full or partial color (white light) image, full or partial desaturated white light image, an enhanced colored image, an overlay (e.g., fluorescence with any other image), a composite image (e.g., fluorescence incorporated into another image) which may have various colors, various levels of desaturation or various ranges of a color to highlight/visualize certain features of interest. Processing of the images may be further performed for further visualization and/or other analysis (e.g., quantification). The lymph nodes and lymphatic vessels may be visualized (e.g., intraoperatively, in real time) using fluorescence imaging systems and methods according to the various examples for ICG and SLNs alone or in combination with a gamma probe (Tc-99m) according to American Society of Breast Surgeons (ASBrS) practice guidelines for SLN biopsy in breast cancer patients. Fluorescence imaging for LNs may begin from the site of injection by tracing the lymphatic channels leading to the LNs in the axilla. Once the visual images of LNs are identified, LN mapping and identification of LNs may be done through incised skin, LN mapping may be performed until ICG visualized nodes are identified. The method of LN mapping per se may exclude any surgical step. For comparison, mapping with isosulfan blue may be performed until ‘blue’ nodes are identified. LNs identified with ICG alone or in combination with another imaging technique (e.g., isosulfan blue, and/or Tc-99m) may be labeled to be excised. Subject may have various stages of breast cancer (e.g., IA, IB, IIA).

In some variations, such as for example, in gynecological cancers (e.g., uterine, endometrial, vulvar and cervical malignancies), ICG may be administered interstitially for the visualization of lymph nodes, lymphatic channels, or a combination thereof. When injected interstitially, the protein binding properties of ICG cause it to be rapidly taken up by the lymph and moved through the conducting vessels to the SLN. ICG may be provided for injection in the form of a sterile lyophilized powder containing 25 mg ICG (e.g., 25 mg/vial) with no more than 5.0% sodium iodide. ICG may be then reconstituted with commercially available water (sterile) for injection prior to use. According to an example, a vial containing 25 mg ICG may be reconstituted in 20 ml of water for injection, resulting in a 1.25 mg/ml solution. A total of 4 ml of this 1.25 mg/ml solution is to be injected into a subject (4×1 ml injections) for a total dose of ICG of 5 mg per subject. The cervix may also be injected four (4) times with a 1 ml solution of 1% isosulfan blue 10 mg/ml (for comparison purposes) for a total dose of 40 mg. The injection may be performed while the subject is under anesthesia in the operating room. In some variations the ICG dose (mg) in gynecological cancer sentinel lymph node detection and/or mapping may range from about 0.1 mg to about 5 mg depending on the route of administration. In some variations, the ICG does may be about 0.1 mg to about 0.75 mg, about 0.75 mg to about 1.5 mg, about 1.5 mg to about 2.5 mg, about 2.5 mg to about 5 mg. The route of administration may be for example cervical injection, vulva peritumoral injection, hysteroscopic endometrial injection, or a combination thereof. In order to minimize the spillage of isosulfan blue or ICG interfering with the mapping procedure when LNs are to be excised, mapping may be performed on a hemi-pelvis, and mapping with both isosulfan blue and ICG may be performed prior to the excision of any LNs. LN mapping for Clinical Stage I endometrial cancer may be performed according to the NCCN Guidelines for Uterine Neoplasms, SLN Algorithm for Surgical Staging of Endometrial Cancer; and SLN mapping for Clinical Stage I cervical cancer may be performed according to the NCCN Guidelines for Cervical Neoplasms, Surgical/SLN Mapping Algorithm for Early-Stage Cervical Cancer. Identification of LNs may thus be based on ICG fluorescence imaging alone or in combination or co-administration with for a colorimetric dye (isosulfan blue) and/or radiotracer.

Visualization of lymph nodes may be qualitative and/or quantitative. Such visualization may comprise, for example, lymph node detection, detection rate, anatomic distribution of lymph nodes. Visualization of lymph nodes according to the various examples may be used alone or in combination with other variables (e.g., vital signs, height, weight, demographics, surgical predictive factors, relevant medical history and underlying conditions, histological visualization and/or assessment, Tc-99m visualization and/or assessment, concomitant medications). Follow-up visits may occur on the date of discharge, and subsequent dates (e.g., one month).

Lymph fluid comprises high levels of protein, thus ICG can bind to endogenous proteins when entering the lymphatic system. Fluorescence imaging (e.g., ICG imaging) for lymphatic mapping when used in accordance with the methods and systems described herein offers the following example advantages: high-signal to background ratio (or tumor to background ratio) as NIR does not generate significant autofluorescence, real-time visualization feature for lymphatic mapping, tissue definition (i.e., structural visualization), rapid excretion and elimination after entering the vascular system, and avoidance of non-ionizing radiation. Furthermore, NIR imaging has superior tissue penetration (approximately 5 to 10 millimeters of tissue) to that of visible light (1 to 3 mm of tissue). The use of ICG for example also facilitates visualization through the peritoneum overlying the para-aortic nodes. Although tissue fluorescence can be observed with NIR light for extended periods, it cannot be seen with visible light and consequently does not impact pathologic evaluation or processing of the LN. Also, florescence is easier to detect intraoperatively than blue staining (isosulfan blue) of lymph nodes. In other variations, the methods, dosages or a combination thereof as described herein in connection with lymphatic imaging may be used in any vascular and/or tissue perfusion imaging applications.

Tissue perfusion relates to the microcirculatory flow of blood per unit tissue volume in which oxygen and nutrients are provided to and waste is removed from the capillary bed of the tissue being perfused. Tissue perfusion is a phenomenon related to but also distinct from blood flow in vessels. Quantified blood flow through blood vessels may be expressed in terms that define flow (i.e., volume/time), or that define speed (i.e., distance/time). Tissue blood perfusion defines movement of blood through micro-vasculature, such as arterioles, capillaries, or venules, within a tissue volume. Quantified tissue blood perfusion may be expressed in terms of blood flow through tissue volume, namely, that of blood volume/time/tissue volume (or tissue mass). Perfusion is associated with nutritive blood vessels (e.g., micro-vessels known as capillaries) that comprise the vessels associated with exchange of metabolites between blood and tissue, rather than larger-diameter non-nutritive vessels. In some examples, quantification of a target tissue may include calculating or determining a parameter or an amount related to the target tissue, such as a rate, size volume, time, distance/time, and/or volume/time, and/or an amount of change as it relates to any one or more of the preceding parameters or amounts. However, compared to blood movement through the larger diameter blood vessels, blood movement through individual capillaries can be highly erratic, principally due to vasomotion, wherein spontaneous oscillation in blood vessel tone manifests as pulsation in erythrocyte movement. In some examples, blood flow and tissue perfusion imaging described herein in connection with the systems and methods may be used to image tumor tissue and differentiate such tissue from other tissue.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated. For the purpose of clarity and a concise description features are described herein as part of the same or separate examples, however, it will be appreciated that the scope of the invention may include examples having combinations of all or some of the features described.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A system for acquiring surgical imaging data, comprising:
a surgical imaging device;
an imaging controller; and
a communication cable for connecting the surgical imaging device to the imaging controller, comprising:
a distal end for connecting with the surgical imaging device;
a proximal end for connecting with the imaging controller;
a single conductor extending between the distal end and the proximal end of the communication cable, the single conductor configured to:
transmit surgical imaging data from the surgical imaging device to the imaging controller, and
transmit a control signal and power from the imaging controller to the surgical imaging device.

2. The system of claim 1, wherein the surgical imaging data comprises at least one of pixel data and voxel data.

3. The system of claim 1, wherein the surgical imaging data comprises data acquired by a plurality of sensors of the surgical imaging device.

4. The system of claim 1, wherein the control signal is a first control signal, and wherein the single conductor is further configured to transmit a second control signal from the surgical imaging device to the imaging controller.

5. The system of claim 1, wherein the surgical imaging data, the control signal, and the power are transmitted according to CoaXPress protocol.

6. The system of claim 1, wherein the single conductor is configured to transmit the surgical image data at around or over 12 gigabits per second.

7. The system of claim 1, wherein the single conductor is configured to transmit the control signal at around or over 40 megabits per second.

8. The system of claim 1, wherein the single conductor is insulated, the communication cable further comprising a cable shielding wrapped circumferentially around the insulated single conductor.

9. The system of claim 8, wherein the cable shielding is configured to provide ground to the power.

10. The system of claim 1, wherein the single conductor is made of at least copper.

11. The system of claim 1, wherein the communication cable comprises a first connector at the proximal end.

12. The system of claim 1, wherein the communication cable comprises a second connector at the distal end.

13. The system of claim 1, wherein the distal end of the communication cable is connected to a transmitter of the surgical imaging device.

14. The system of claim 13, wherein the transmitter is configured to transmit the surgical imaging data according to the CoaXPress protocol.

15. The system of claim 13, wherein the transmitter includes a clock.

16. The system of claim 1, wherein the proximal end of the communication cable is connected to a receiver of the imaging controller.

17. The system of claim 16, wherein the receiver is housed in a connector board of the imaging controller.

18. The system of claim 16, wherein the receiver is configured to receive the surgical imaging data according to the CoaXPress protocol.

19. The system of claim 16, wherein the receiver is configured to perform equalization on the received surgical imaging data.

20. The system of claim 1,
wherein the communication cable is a first cable and the surgical imaging data is a first set of surgical imaging data, and
wherein the system further comprises:
a second cable for connecting the surgical imaging device to the imaging controller, wherein the second cable is configured to transmit a second set of surgical imaging data from the surgical imaging device to the imaging controller.

21. A surgical imaging device configured to be connected to a communication cable, comprising:
a camera component for obtaining surgical imaging data;
a transmitter configured to be connected to the communication cable, the transmitter configured to simultaneously:
transmit the surgical imaging data via a single conductor of the communication cable, and
receive a control signal and power via the single conductor of the communication cable.

22. The device of claim 21, wherein the transmitter is configured to transmit the surgical imaging data according to the CoaXPress protocol.

23. The device of claim 21, wherein the transmitter includes a clock.

24. The device of claim 21, wherein the control signal is a first control signal, and wherein the transmitter is configured to transmit a second control signal via the single conductor of the communication cable.

25. A imaging controller configured to be connected to a communication cable, comprising:
a control component for generating a control signal; and
a receiver configured to be connected to the communication cable, the receiver configured to simultaneously:
receive surgical imaging data via a single conductor of the communication cable, and
transmit the control signal and power via the single conductor of the communication cable.

26. The imaging controller of claim 25, wherein the receiver is housed in a connector board of the imaging controller.

27. The imaging controller of claim 25, wherein the receiver is configured to receive the surgical imaging data according to the CoaXPress protocol.

28. The imaging controller of claim 25, wherein the receiver is configured to perform equalization on the received surgical imaging data.

29. The imaging controller of claim 25, wherein the control signal is a first control signal, and wherein the receiver is configured to receive a second control signal via the single conductor of the communication cable.
